(19) United States
(12) Patent Application Publication
(10) Pub. No.: US 2023/0018939 A1
(43) Pub. Date: Jan. 19, 2023

(54) ON-LINE ULTRA PERFORMANCE HYDROPHOBIC INTERACTION CHROMATOGRAPHY FOR MONITORING AT LEAST ONE PRODUCT ATTRIBUTE

(71) Applicants: Mark A. Brower, Kenilworth, NJ (US); Jayesh Desai, Kenilworth, NJ (US); Nuno Jorge Dos Santos Pinto, Kenilworth, NJ (US); Zachary D. Dunn, Kenilworth, NJ (US); Adrian Gospodarek, Kenilworth, NJ (US); Xiaoqing Hua, Kenilworth, NJ (US); Bhumit A. Patel, Kenilworth, NJ (US); Douglas D. Richardson, II, West Point, PA (US); Merck Sharp & Dohme LLC, Rahway, NJ (US)

(72) Inventors: Mark A. Brower, Bound Brook, NJ (US); Jayesh Desai, Paterson, NJ (US); Nuno Jorge Dos Santos Pinto, Newark, NJ (US); Zachary D. Dunn, Harrison, NJ (US); Adrian Gospodarek, Cranford, NJ (US); Xiaoqing Hua, Piscataway, NJ (US); Bhumit A. Patel, Edison, NJ (US); Douglas D. Richardson, II, Collegeville, PA (US)

(73) Assignee: Merck Sharp & Dohme LLC, Rahway, NJ (US)

(21) Appl. No.: 17/778,999

(22) PCT Filed: Nov. 20, 2020
(86) PCT No.: PCT/US2020/061435

§ 371 Date: May 23, 2022

Related U.S. Application Data
(60) Provisional application No. 62/940,588, filed on Nov. 26, 2019.
(60) Provisional application No. 62/981,707, filed on Feb. 26, 2020.

(51) Int. Cl. G01N 30/88; G01N 30/24; G01N 30/14

(52) U.S. Cl. CPC G01N 30/88; G01N 30/24; G01N 30/14; G01N 2030/027

(57) ABSTRACT

The present invention relates to a method of measuring at least one product quality attributes, including critical quality attributes (e.g., oxidation, C-terminal lysine, aggregation, N-terminal glutamine cyclization.) by an online liquid chromatography system, wherein the liquid chromatography system is running a hydrophobic interaction chromatography (HIC) method.

### BACKGROUND OF THE INVENTION

In the biopharma industry, continuous manufacturing has the potential to increase efficiency, flexibility, agility, and robustness of manufacturing by reducing the number of steps and holds, utilizing smaller equipment and facilities, improving product quality, and enabling real-time release of manufacturing batches and pharmaceutical product. See also, “Biomanufacturing Technology Roadmap—Continuous Downstream Processing for Biomanufacturing”, Biophorum, www.biophorum.com/continuous-downstream-processing-for-biomanufacturing-an-industry-review.

Process analytical technology (PAT) is a mechanism to design, analyze, and control pharmaceutical manufacturing processes through the measurement of critical process parameters which affect product quality attributes including critical quality attributes (CQAs) that ensure final product quality. PAT allows for understanding of the manufacturing process by defining critical processing parameters (CPPs) and accordingly monitoring them in a timely manner (in-line, at-line, or on-line) and adjusting process parameters if necessary, allowing for greater efficiency in testing, reducing over-processing, enhancing consistency, and minimizing rejected batches.

The objective for PAT implementation includes: better process understanding, improved yield because of prevention of the reprocessing, reduction in the production cycle time by using on-line/at-line or in-line measurements and control, improved efficiency from conversion of the batch process into continuous processing, cost reduction, and real-time release of the batches.

At-line process analytical technology, including cell counters or biochemistry analyzers, are physically located next to the process and requires manual sampling. On-line process analytical technology, which includes aseptic sampling or on-line liquid chromatography, are physically connected directly to the process and include automatic sampling where the sampling never returns to the process due to potential contamination risk to the purification batch.

In-line process analytical technology is physically in contact with or in the flow of the process. In-line process analytical technology requires no sampling and provides continuous data. Such examples of in-line process analytical technology includes capacitance, spectroscopy, light scattering, and sensors.

A typical downstream purification process for an antibody includes affinity chromatography, virus inactivation, one or two polishing purification steps, and subsequent filtration steps to achieve drug substance.

The PATROL® UPLC Process Analysis System designed by Waters provides access to real-time, chromatographic-quality analysis for in-process samples. This PAT tool provides streaming CQAs and therefore increases efficiencies, reduces costs, ensures final product quality, improves the yield, shortens the overall product development lifecycle, and builds a stronger scientific knowledge base for all products.

Although monoclonal antibodies (mAbs) are a very stable class of therapeutic proteins, and normally exhibit good pharmacokinetic profiles in patients, they are still susceptible to a variety of post-translational modifications (PTMs) during production and storage. The observed PTMs include glycosylation, N-terminal glutamine cyclization and C-terminus lysine cleavage, deamidation, isomerization, and oxidation. Among these modifications, oxidation is commonly detected and results from reaction of reactive oxygen species with solvent-exposed amino acid residues including methionine, tyrosine, tryptophan, and cysteine residues. Oxidation of amino acids in the complementarity determining regions (CDRs) and the fragment crystallizable region (Fc region) could potentially impact biological efficacy, clearance, safety and immunogenicity of mAbs. Oxidation has been shown to affect antibody binding to Fc receptors and antigens, and to impact mAb stability and half-life. Therefore, it is critical to measure and monitor oxidation during different-stages of drug development and production.

Monitoring the levels of PTMs (e.g., oxidation, C-terminal lysine heterogeneities, etc) relies almost exclusively on liquid chromatography (LC) and/or LC-mass spectrometry (LC-MS) based methodologies, which are essential for characterizing biologics because of the sensitivity, resolution, selectivity, and specificity of the technique. Specifically, reduced peptide mapping via reversed phase (RP) LC coupled with MS is a very effective approach for identifying the modification sites, and monitoring oxidation and other PTMs. However, it has some limitations as the sample preparation process for RP LC-MS is lengthy, and in some cases the chromatographic conditions such as high temperature, organic solvents, and acidic pH conditions could induce oxidation artifacts. Although protein A chromatography have been used for analysis of antibody oxidation in some cases, these methods require long chromatographic run times, and have the limited power for separating oxidized protein variants.

In addition, ion-exchange chromatography (IEX), isoelectric focusing (IEF), capillary isoelectric focusing (cIEF), and imaged capillary isoelectric focusing (icIEF) have been used to analyze C-terminal lysine species, N-terminal glutamine cyclization, deamidation, and isomerization. However, these methods either require sample preparation, have extensive run times, or there is a limitation where the resolution is not adequate to separate one charged species from another.

Along with PTMs, immunogenicity can also be impacted by aggregation of mAbs, which can affect product quality, safety, and efficacy. Since mAbs have a tendency to aggregate during manufacturing, shipping, and storage, aggregation is typically classified as a CQA.

Although C-terminal lysine variation and N-terminal glutamine cyclization are not known to impact antibody function, it is critical to monitor to ensure that product quality remains consistent throughout the manufacturing process. C-terminal lysine variation and N-terminal glutamine cyclization are normally detected via IEX, and lack of control will potentially impact release specifications. Conversely, the consequence of oxidation and aggregation could potentially result in the loss of biological activity, increase of immunogenicity, bulk or product shelf life, and/or release specification failure.

Monitoring PTMs like oxidation during the development of mAb is critical for the demonstration of product consistency, efficacy and/or stability. The oxidation of solvent exposed methionine residue especially in the antigen-binding fragment (Fab) region and Fc region is a major concern as it can lead to immunogenicity, reduced binding affinity and short half-life of the biotherapeutics.

HIC is a method that separates proteins, including monoclonal antibodies based on molecular hydrophobicity. The HIC mobile phase usually consists of a salting-out agent, which at high salt concentration retains the protein by increased hydrophobic interactions between the protein and the stationary phase. Bound proteins are eluted by decreasing the salt concentration. HIC mobile phases typically contain little or no organic solvent at physiological pH level, which allows the protein to preserve its native conformation. Therefore, separation of conformational differences under the native state of the protein may also be achieved.

### SUMMARY OF THE INVENTION

While the use of online LC for PAT is known in the art, the method of applying an on-line liquid chromatography system running a HIC analytical method is not known.

For a schematic of at-line, in-line and on-line, refer to FIG. 11 (Karle, et al., Microfluidic solutions enabling continuous processing and monitoring of biological samples: A review, Analytica Chimica Acta, Vol. 929, 27 Jul. 2016, pages 1-22).

The present invention comprises a method of measuring at least one product attribute (e.g., oxidation, aggregation, C-terminal lysine and N-terminal glutamine cyclization) by an online LC system, wherein the LC system is running a HIC method.

In certain embodiments, the present invention comprises a method of measuring at least one product attribute of a monoclonal antibody process intermediate by an online LC system, wherein the LC system is running a HIC method. In a certain embodiment, the process intermediate is located in the bioreactor or protein A feed. In another embodiment, the online HIC analytical method measures at least one product attribute of a process intermediate in a downstream purification process. In another embodiment, the process intermediate is protein A feed, protein A product, viral inactivation feed, viral inactivation product, AEX load, AEX product, CEX load, CEX product, HIC load, HIC product, viral filtration feed, ultrafiltration or diafiltration feed, bulk drug substance (DS), or bulk drug product (DP).

In a particular embodiment, the present invention comprises a method of measuring at least one product attribute of a monoclonal antibody process intermediate by an online HIC system connected to a purification process. In a particular embodiment, the present invention comprises the LC system connected online to the purification process aseptically. In a particular embodiment, the present invention comprises the LC system connected online to the purification process through direct aseptic connection.

In a particular embodiment, the present invention comprises an online HIC analytical method measuring at least one product attribute of a monoclonal antibody process intermediate. In a further embodiment, the process intermediate is in a bioreactor or protein A feed. In a further embodiment, the present invention comprises an online HIC measuring at least one product attribute of a process intermediate in a downstream purification process. In a further embodiment, the process intermediate is protein A feed, protein A product, viral inactivation feed, viral inactivation product, AEX load, AEX product, CEX load, CEX product, HIC load, HIC product, viral filtration feed, ultrafiltration or diafiltration feed, or bulk DS.

In a particular embodiment, the present invention comprises a HIC analytical method measuring at least one product attribute of a monoclonal antibody process intermediate, wherein the HIC is connected online to the purification process, and wherein the purification process is operated in an integrated, connected, and continuous manner from a perfusion bioreactor. An integrated connected and continuous process refers to the fact that multiple unit operations are either directly connected or connected through a surge vessel (operating as a continuously stirred tank reactor (CSTR)) to one another and the unit operations are coordinated through a master control system. For example, an integrated connected and continuous process refers to the 2 or more multiple unit operations connected directly or connected through a surge vessel. It is not a requirement that all downstream unit operations are connected in this manner.

In a particular embodiment, the present invention comprises an online HIC analytical method measuring at least one product attribute of a process intermediate during continuous purification of a monoclonal antibody. In a further embodiment, the process intermediate is in a bioreactor or is protein A feed. In a further embodiment, the present invention comprises an online HIC measuring at least one product attribute of a process intermediate in a downstream purification process. In a further embodiment, the present invention comprises an online HIC measuring at least one product attribute of a process intermediate selected from the group consisting of: protein A feed, protein A product, viral inactivation feed, viral inactivation product, AEX load, AEX product, CEX load, CEX product, HIC load, HIC product, viral filtration feed, ultrafiltration or diafiltration feed, or bulk DS. One embodiment of the present invention comprises an online HIC analytical method measuring at least one product attribute of a process intermediate during continuous purification of a monoclonal antibody, wherein the process intermediate is downstream of protein A.

In a particular embodiment, the present invention comprises an online HIC analytical method testing at least one product attribute of a process intermediate at specified intervals for monitoring the product attribute at various levels or ranges during the protein A purification step of a monoclonal antibody, wherein the process intermediate is protein A product.

In a particular embodiment, the HIC is connected online to the purification process aseptically to prevent the risk of contamination into the product stream.

In a particular embodiment, the present invention comprises an online HIC analytical method comprising the steps of:
(a) obtaining a sample from the purification process,
(b) automatically injecting said sample onto a HIC column, and
(c) performing a real-time analytical method measuring at least one product attribute level.

In a particular embodiment, the present invention comprises an online HIC analytical method comprising the steps of:
(a) obtaining a sample from the purification process,
(b) automatically injecting said sample onto a HIC column,
(c) performing a real-time analytical method measuring at least one product attribute, and
(d) repeating steps (a) and (b) for the duration of the purification process, while analyzing and completing real-time process decisions to continue, stop or segregate the downstream purification process.

In a particular embodiment, the present invention comprises an online HIC analytical method comprising the steps of:
(a) obtaining a sample from the purification process,
(b) automatically injecting said sample onto a HIC column,
(c) performing a real-time analytical method measuring at least one product attribute level, and
(d) repeating steps (a) and (b) for the duration of the purification process, while analyzing and completing real-time process decisions to continue, stop or segregate the downstream purification process,
(e) optionally making process adjustments to the upstream bioreactor production process or downstream purification process based on the results obtained during real-time monitoring for the product attribute(s) subject to testing,
(f) wherein the process adjustments comprise continuing or stopping the downstream purification process, or segregating the harvested cell culture fluid (HCCF) or the downstream purification product stream, and
(g) wherein segregating the HCCF or the downstream product stream prevents contamination to HCCF or the downstream product stream collected prior to the process adjustment.

In a particular embodiment, the present invention comprises an online HIC analytical method measuring at least one product attribute level wherein the method comprises:
(a) obtaining a sample from the purification process,
(b) injecting the sample onto a HIC column,
(c) eluting the sample from the column based on salt concentration or pH,
(d) obtaining a value for a product quality attribute.

In a particular embodiment, the present invention comprises injecting a sample onto a HIC column, wherein the sample is a volume. In a further embodiment, the volume is fixed.

In a particular embodiment, the present invention comprises injecting a sample onto a HIC column, wherein the sample is a concentration. In a further embodiment, the concentration is fixed.

In a particular embodiment of the present invention, the sample is taken from the affinity chromatography purification step. In a subembodiment of the present invention, the sample is taken from the protein A chromatography purification step. In a subembodiment of the present invention, the sample is taken from the ion exchange chromatography purification step. In a subembodiment of the present invention, the sample is taken from the AEX purification step. In a subembodiment of the present invention, the sample is taken from the CEX purification step. In a subembodiment of the present invention, the sample is taken from the mixed mode purification step.

In a particular embodiment of the present invention, the sample is taken from the purification step product. In a subembodiment of the present invention, the purification step product is protein A product, viral inactivation feed, viral inactivation product, AEX load, AEX product, CEX load, CEX product, HIC load, HIC product, viral filtration feed, ultrafiltration or diafiltration feed, or bulk DS.

In a particular embodiment of the present invention, the on-line HIC process does not comprise a manual dilution. In one embodiment of the present invention, the on-line HIC process is automated.

The on-line HIC process of the present invention provides real-time data to make real-time decisions to ensure product quality of the processing mAb batch.

mAbs are susceptible to oxidation during production, storage and shipping. Oxidation of methionine or tryptophan residues in CDRs have been linked to decreased or loss of bioactivity of mAbs. Therefore, it is critical to monitor oxidation to confirm the stability and clinical efficacy of mAb products.

One aspect of the present invention comprises measuring or monitoring a product attribute such as oxidation, aggregation, C-terminal lysine and N-terminal glutamine cyclization.

In a further embodiment of the present invention, the product attribute is oxidation. In another embodiment of the present invention, the product attribute is aggregation. In a further embodiment of the present invention, the product quality attribute is C-terminal lysine. In yet a further embodiment of the present invention, the product quality attribute is N-terminal glutamine cyclization.

For any particular product attribute, the levels can vary among cell lines and product characteristics. Monitoring the product attribute(s) provides a baseline at the start of the purification process and while monitoring during the process, if there is a fluctuation in the level, the operator can make appropriate adjustments to the process in real-time if there is a fluctuation in the level of the product attribute. The levels for these product attributes can provide an indication of when to quarantine specific portions of the product to ensure product quality. A person of ordinary skill in the art can calculate based on residence time how long it takes to complete purification and when the compromised product will have eluted from a specific purification step.

Other aspects of the present invention comprise the use of an on-line and/or at-line LC system, wherein the online and/or at-line LC system is running a HIC analytical method, and wherein the analytical method is measuring at least one product quality attribute selected from the group comprising: i) oxidation, ii) aggregation, iii) C-terminal lysine and/or N-terminal glutamine cyclization. Another aspect of the present invention comprises the use of measuring at least one product attribute of a process intermediate during the monoclonal antibody purification process by an online LC system, wherein the LC system is running a HIC method.

In a certain embodiment, the process intermediate is located in the bioreactor or protein A feed. In another embodiment, the online HIC analytical method measures at least one product quality attribute of a process intermediate in a downstream purification process. In another embodiment, the process intermediate is protein A feed, protein A product, viral inactivation feed, viral inactivation product, AEX load, AEX product, CEX load, CEX product, HIC load, HIC product, viral filtration feed, ultrafiltration or diafiltration feed, or bulk DS.

In a particular embodiment, the present invention comprises the use of an online HIC analytical method measuring at least one product attribute of a process intermediate during continuous purification of a mAb.

In a particular embodiment, the present invention comprises the use of an online HIC analytical method measuring at least one product attribute of a process intermediate at specified intervals while monitoring the product attribute at various levels or ranges of a mAb during the protein A purification step.

In a particular embodiment, the process intermediate is protein A product.

In a particular embodiment, the present invention comprises a method of running a HIC, wherein the HIC column can be operated in a bind-elute mode or a flow-through mode.

### BRIEF DESCRIPTION OF THE DRAWINGS

FIG. 1 shows a HIC profile of an IgG4 monoclonal antibody via online and atline injection.

FIG. 2 shows HIC profiles of an IgG4 monoclonal antibody tested at various time points demonstrating different oxidation levels via online injection.

FIG. 3 shows a scatter plot for control material being stressed with t-BHP resulting in different oxidation levels using an online HIC method

FIG. 4 is a scatter plot showing total oxidation percent for protein A product.

FIG. 5 is a scatter plot showing total oxidation percent for filtered neutralized viral inactivation product.

FIGS. 6A and 6B are HIC profiles that show an overlay of injections displaying the presence and absence of C-terminal lysine species. FIG. 6B is a closeup image of FIG. 6A.

FIG. 7 shows an IgG4 monoclonal antibody generated under different upstream cell culture conditions.

FIG. 8 shows the identity of the pre-main peak of HIC profile of mAb 1 to be N-terminal pyroglutamate.

FIG. 9 is a flowchart representing the automated workflow for online Pro-A HIC analysis of a bioreactor sample.

FIG. 10A represents two chromatography profiles showing the results of an online analytical Pro-A HIC to detect oxidation species from Protein A feed.

FIG. 10B represents two chromatography profiles showing the results of an online analytical Pro-A HIC to detect oxidation species from bioreactor samples.

FIG. 11 is a schematic of the type of monitoring principles employed for continuous microfluidic monitoring systems.

FIG. 12 shows the percent oxidation of samples taken during a continuous manufacturing process.

FIGS. 13A and 13B show the on-line LC system monitoring oxidation during a continuous batch and measuring three different sample locations, (FNVIP, AEXP, and DS) and comparing the results through offline measurements.

FIGS. 14A and 14B show the on-line LC system measuring a perturbation experiment where oxidized and non-oxidized material were being exchanged through various unit operations.

FIGS. 15A and 15B show representative HIC profiles of mAb 2 with different aggregates levels to elucidate the separation of aggregates via offline injection.

### DETAILED DESCRIPTION OF THE INVENTION

### Definitions

In order that the present invention may be more readily understood, certain terms are first defined.

As used throughout the specification and in the appended claims, the singular forms “a,” “an,” and “the” include the plural reference unless the context clearly dictates otherwise. Reference to “or” indicates either or both possibilities unless the context clearly dictates one of the indicated possibilities. In some cases, “and/or” was employed to highlight either or both possibilities.

The term “product”, as used herein refers to a protein of interest, which may be present in the context of a sample comprising one or more process-related impurities and/or product-related substances. In certain embodiments, the product, i.e., the protein of interest, is an antibody or antigen binding fragment thereof.

The term ‘online’ or ‘on-line’ includes sampling to a chromatography unit that is physically connected directly to the process and includes automatic sampling (including when instructed by an operator) where the sampling never returns to the process due to potential contamination risk to the purification batch.

The term ‘at-line’ or ‘atline’ includes sampling to a chromatography unit that is physically located next to the process and requires manual sampling.

The term ‘in-line’ or ‘inline’ includes sampling physically in contact with or in the flow the of the process.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “antibody”, as used herein, also includes alternative antibody and antibody-like structures, such as, but not limited to, dual variable domain antibodies (DVD-Ig).

The term “antigen-binding portion” of an antibody (or “antibody portion”) includes fragments of an antibody that retain the ability to specifically bind to an antigen (e.g., hIL-12, hTNFα, or hIL-18). It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody may be part of a larger immunoadhesion molecule, formed by covalent or non-covalent association of the antibody with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The term ‘antibody’ refers to any form of antibody that exhibits the desired biological activity. Thus, it is used in the broadest sense and specifically covers, but is not limited to, monoclonal antibodies (including full length monoclonal antibodies), polyclonal antibodies, bispecific antibodies, humanized, fully human antibodies, and chimeric antibodies.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

The term ‘liquid chromatography’ or ‘chromatography’ is a chromatographic technique that is useful for separating ions or molecules that are dissolved in a solvent. The separation occurs based on the interactions of the sample with mobile and stationary phases. Liquid chromatography features a liquid mobile phase which slowly filters down through the solid stationary phase.

Hydrophobic interaction chromatography (HIC) is a method that separates proteins, including monoclonal antibodies based on molecular hydrophobicity. Hydrophobic groups on the protein interact with hydrophobic groups of the stationary phase or the membrane. In certain embodiments, the more hydrophobic a protein is, the stronger it will interact with the stationary phase or the membrane. Thus, HIC steps, such as those disclosed herein, can be used to remove a variety of process-related impurities (e.g., DNA) as well as product-related species (e.g., high and low molecular weight product-related species, such as protein aggregates and fragments).

The interaction between hydrophobic proteins and a HIC resin is influenced significantly by the presence of certain salts in the running buffer. A high salt concentration enhances the interaction while lowering the salt concentration weakens the interaction. The HIC mobile phase usually consists of a salting-out agent, which at high concentration retains the protein by increasing hydrophobic interaction between the protein and the stationary phase. Bound proteins are eluted by decreasing the salt concentration. HIC mobile phases typically contain little or no organic solvent at physiological pH levels, which allows the protein to preserve its native structure. Therefore, conformational changes in the native form of the protein may be analyzed.

HIC can be used for capture, intermediate purification, or polishing steps. As samples should be in a high salt concentration to promote hydrophobic interaction, HIC is well-suited for capture steps after sample cleanup by ammonium sulfate precipitation or for intermediate steps directly after an ion exchange separation.

The present invention features a method for measuring product quality attributes through an on-line and/or at-line LC system running a HIC method. In performing a HIC method, the sample mixture is contacted with the chromatographic material (e.g., stationary phase, membrane, etc) suitable for separation based on hydrophobic interactions. A chromatography column, or other known technique in the art, can be employed and prepared in an appropriate buffer solution. Once the sample of the process intermediate is fed to the chromatographic apparatus (e.g., an antibody is contacted to the chromatography material to induce separation), the protein of interest, process-related impurities, and/or product related substances are separated from the chromatographic material by washing the material and collecting the fractions from the column. The chromatographic material can be subjected to one or more wash steps, if desired. The chromatographic material can then be contacted with a solution designed to desorb any components of the solution that have bound to the chromatographic material.

Analytes bind to the weakly hydrophobic stationary phase in the presence of high salt concentrations and elute off the column as the salt concentration decreases. HIC typically preserves the biological activity of the protein, which is useful for biological analysis such as antigen binding, Fc binding and cell-based potency assays. In addition, HIC typically provides separation with little to no carryover. Due to these benefits, HIC is not only used for variants analysis of therapeutic proteins including mAbs but also has been widely used as a purification method for biotherapeutic products.

A HIC column or membrane device can be operated in a bind-elute mode, a flow-through mode, or a hybrid mode wherein the product exhibits binding to the chromatographic material yet can be washed from the column using a buffer that is the same or substantially similar to the loading buffer. For flow-through, process-related impurities, such as HCPs, and product-related substances, such as aggregates and fragments, will, depending the particular HIC material employed, bind to the HIC media while product flows through the column. A hybrid mode, in contrast, can involve the use of an HIC media that allows for the product to be immobilized on the chromatographic support in the presence of a loading buffer, but then be removed by successive washes of buffer identical to or substantially similar to the loading buffer, for example, but not by way of limitation where the salt concentration is adjusted within about 20% of the concentration of the loading buffer. In certain embodiments, a step-wise or linear change in wash conductivity can be used. In the context of this hybrid strategy, process-related impurities and product-relates substances will either bind to the chromatographic material or flow through with a profile distinct from the protein of interest. After loading, the column can be regenerated with water and cleaned with caustic solution to remove the bound impurities before next use.

HIC resins can include a differentiating range in hydrophobicity suitable for bind/elute and flow-through applications (e.g., POROS Ethyl, POROS Benzyl, POROS Benzyl Ultra, Proteomix® HIC-NP (non-porous polystyrenedivinylbenzene (PS/DVB) beads)). The resins can be utilized at lower salt concentrations for the purification of a wide variety of biotherapeutic products.

HIC separation is based on the interaction between the hydrophobic ligands on HIC stationary phase and/or membrane and the hydrophobic surfaces on proteins. For selective elution (desorption), the salt concentration can be lowered gradually via gradient elution or can be eluted isocratically, with the sample components eluting in the order of their hydrophobicity with the more hydrophobic components eluting later. The type and concentration of salt used to dissolve the sample, as well as that used in the HIC mobile phases, will influence the separation. Changing the type of salt and the starting salt concentration can change the selectivity of the obtained separation.

Hydrophobic interactions are strongest at high salt concentration (and hence the ionic strength of the anion and cation components is maximized); therefore, this form of separation during downstream purification is conventionally performed following salt elution step, e.g., the type of elution step typically used in connection with ion exchange chromatography. Adsorption of the antibody to a HIC column is favored by high salt concentrations, but the actual concentrations can vary over a wide range depending on the nature of the protein of interest, salt type and the particular HIC ligand chosen. For example, and not by way of limitation, the salt concentrations shown to be effective in aggregate reduction are in the range of 80 mM-1000 mM, depending on the salt type and HIC adsorbent. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba²⁺; Ca²⁺; Mg²⁺; Li⁺; Cs⁺; Na⁺; K⁺; Rb⁺; NH₄⁺, while anions may be ranked in terms of increasing chaotropic effect as PO₄³⁻; SO₄²⁻; CH₃CO₃⁻; Cl⁻; Br⁻; NO₃⁻; ClO₄⁻; I⁻; SCN⁻.

Additionally, the pH of the mobile phase can affect the charge of the protein which contributes to the overall hydrophobicity of the sample. Therefore, adjusting the pH may result in a different separation profile. The slope of the gradient affects the resolution and can be adjusted accordingly. In some cases, slower flow rates may improve the resolution. Temperature affects the hydrophobic interactions between the protein and the stationary phase and therefore can be adjusted accordingly to optimize separation efficiency.

HIC can be performed with buffers known in the art. For example, mobile phase A and mobile phase B can be varying salts used in HIC separation.

Hydrophobic interaction chromatography columns are those known in the art and are used depending on specific attributes(s) or separation.

The term ‘process intermediate’ as used herein, is intended to comprise the mAb throughout the purification process prior to drug product. For example, protein A product is a process intermediate after the sample has been eluted from the protein A column, even if the entire elution cycle of the protein A feed has not been completed. A process intermediate comprises anything downstream in the purification process including but not limited to; protein A feed, protein A product, viral inactivation feed, viral inactivation product, AEX load, AEX product, CEX load, CEX product, HIC load, HIC product, viral filtration feed, ultrafiltration or diafiltration feed, or bulk drug substance. A process intermediate additionally comprises the protein of interest located in the bioreactor, harvested cell culture fluid, and/or clarified cell culture fluid.

The term ‘protein A feed’ as used herein, is intended to comprise the feed material to be loaded onto the protein A column, including but not limited to continuous protein A feed material and in process protein A feed material. The protein A feed can be classified as an upstream or downstream intermediate.

The term ‘protein A product’ as used herein, is intended to comprise the eluate from the column of protein A, including but not limited to, continuous protein A and in process protein A. Protein A product is intended to comprise protein A pooled product.

The term ‘protein A pooled product’ as used herein, is intended to comprise the eluate from at least two protein A product chromatography elution's. The protein A pooled product can be from one protein A column or from more than one protein A column. The protein A pooled product can be from the same protein A purification step, the same protein A purification cycle, or can comprise eluate from more than one cycle of protein A from the same or different columns.

The downstream purification of the invention begins at the separation step when the antibody has been produced using production methods conventional in the art. Once a clarified solution or mixture comprising the antibody has been obtained, separation of the protein of interest from process-related impurities, such as the other proteins produced by the cell, as well as any product-related substances such as charge variants and/or size variants (aggregates and fragments), can be performed using a series of filtration and chromatography steps or techniques including but not limited to a filtration and/or affinity, ion exchange, and/or mixed mode chromatographic step(s). The essence of each of the chromatographic separation methods is that proteins can be caused either to traverse at different rates down a column, achieving a physical separation that increases as they pass further down the column, or to adhere selectively to the separation medium, being then differentially eluted by different solvents. In some cases, the protein of interest is separated from impurities and or product-related substances when the impurities and/or product-related substances specifically adhere to the column and the protein of interest does not, i.e., the protein of interest is washed from the column, while in other cases the protein of interest will adhere to the column, while the impurities and/or product-related substances are washed from the column.

The term ‘downstream of protein A’ comprises the protein A product and the product from any further filtration or purification steps including but not limited to; viral inactivation feed, viral inactivation product, AEX load, AEX product, CEX load, CEX product, HIC load, HIC product, viral filtration feed, ultrafiltration or diafiltration feed, or bulk DS.

A process intermediate comprises anything downstream in the purification process including but not limited to; protein A feed, protein A product, viral inactivation feed, viral inactivation product, AEX load, AEX product, CEX load, CEX product, HIC load, HIC product, viral filtration feed, ultrafiltration or diafiltration feed, or bulk DS.

The term ‘real-time’ as used herein, is intended to comprise immediate results provided by an online HIC system measuring product as it is in the process of purification. The term ‘immediate’, as used herein, is intended to include the time it takes for the duration of the analytical method to execute but with the understanding that it provides real-time data.

The term ‘product quality attribute’ refers to a drug's physical, chemical, biological, or microbiological property or characteristics that could affect the quality of the drug. Manufacturing is designed to control the desired levels of specific product attributes that could affect quality. Product quality attribute(s) include oxidation, aggregation, fragmentation, and other PTMs such as oxidation, C-terminal lysine, deamidation, N-terminal glutamine cyclization, amidation, isomerization and hydroxylation.

The term ‘direct aseptic connection’ as used herein, is a connection that comprises an intermediate sampling device in a system, wherein the sample can be either pushed or pull.

The MAbPAC™ HIC-20 column was a high resolution, silica-based HIC column designed for the separation of mAbs and mAb variants. The Proteomix® HIC-NP-Butyl phase has advantages for biomolecule separations with a wide pH range and high chemical stability. The Proteomix® HIC-NP-Butyl has mobile phase compatibility with aqueous solution, a mixture of water and acetonitrile, acetone, methanol, or THF. The nonporous structure, unique chemistry, and narrow particle distribution offer special selectivity, high-resolution separation of proteins such as mAb, ADC and related protein fragments, DNA and oligonucleotides. Other equivalent columns and mobile phases are acceptable.

Two-dimensional liquid chromatography (2D LC) is a type of chromatographic technique in which the injected sample is separated by passing through two different separation stages. Two chromatographic columns are connected in sequence, and the effluent from the first separation system is transferred onto the second separation. On-line 2D chromatography refers to one or many fractions of the effluent from the first column being transferred (injected) to a second column. (See also, Demystifying Two-Dimensional Liquid Chromatography, The Analytical Scientist, Nov. 19, 2014), (See also, Olivieri, et al., Chapter 3—Experimental Three-way/Second Order Data, Practice Three-Way Calibration, 2014, pages 27-45).

### Abbreviations

AEX Anion Exchange Chromatography
CEX Cation Exchange Chromatography
CPP critical processing parameter
CRS Cell Removal System
CQA critical quality attribute
HCCF Harvested Cell Culture Fluid
HIC Hydrophobic Interaction Chromatography
LC Liquid Chromatography
LCMS Liquid chromatography mass spectrometry
mAb Monoclonal Antibody
MAST Modular Automated Sampling Technology
PAP Protein A Product
PAT Process analytical technology
PTM Posttranslational modification
PDA Photodiode Array
PSM Process Sample Manager
PEEK Poly ether ether ketone
RP-LC-MS Reversed Phase-Liquid chromatography-mass spectrometry
tBHP tert-butyl hydroperoxide
UPLC Ultra performance liquid chromatography
UV ultraviolet

### EXAMPLES

An integrated on-line LC system running a HIC analytical method with automated sampling where the sample is drawn from a location within the process. In this example, the process intermediate tested was protein A product.

The Waters PATROL® UPLC Process Analysis System (Waters, Milford, Mass., USA) was used for on-line sampling drawing the sample from protein A product (PAP). The Waters PATROL® UPLC was equipped with a Process Sample Manager (PSM), Column Manager (Code CMP—2 columns), UV detector, and a Quaternary Pump. The PSM contained a process pump (which obtained material from the process), a sample pump (for filling the injection loop), and a diluent pump (which can dilute the sample 1-100×). The PSM directly injected the sample onto an analytical column for on-line analysis. The PSM also contained a carousel capable of housing manually collected samples at a controlled temperature. These manually collected samples can also be injected at-line through the same fixed loop. The Waters PATROL® UPLC Process Analysis System also contained a PDA detector which is a diode array detector used for obtaining spectral profiles from molecular mixtures or chromatographically separated samples.

### Example 1-3 Day Forced Oxidation

Materials:

Material/Reagent Name, Company Brand Name, Part Number
Sodium Phosphate Monobasic Monohydrate, Fisher BioReagents, BP330-1
Sodium Phosphate dibasic heptahydrate, Sigma Aldrich, S9390-1KG
Ammonium Sulfate, Sigma Aldrich, A2939-1KG
Acetonitrile (ACN), Fisher Chemical, A955-4
Sodium Hydroxide, Sigma Aldrich, 415413-500ML
tert-butyl hydroperoxide (tBHP) Acros Organics, 180342500
70% alcohol, A459-1
Weldable tubing and PEEK tubing of 1/16″ OD×0.020″ ID
MAbPAC HIC20 column, Thermo Fisher Scientific, 088553-088555
Proteomix® HIC Butyl, Sepax Technologies, Inc., 431NP2-4610

Buffers:
Buffers: 5 mM sodium phosphate, pH 7.0 with 2% ACN
Buffers: 5 mM sodium phosphate, 1.5M ammonium sulfate, pH 6.9 with 2% ACN
Buffers: 50 mM sodium phosphate, pH 7.0 with 4% ACN
Buffers: 50 mM sodium phosphate, 1.0M ammonium sulfate, pH 6.9 with 4% ACN

Monoclonal Antibodies:

Antibody (mAb 1, IgG4) was produced in CHO cells and purified via a purification process including Protein A chromatography.

HIC Chromatography Method for On-Line Sampling:

The chromatography system was aseptically connected and sanitized to prevent contamination to the liquid stream during processing. The draw/delivery parameters were set to 1.0 mL/min and sample volume via needle fill was set to 2.0 mL when connected to PAP (or if applicable, reference material). An injection volume of 5 μL Protein A Product (PAP for online injections, and PAP and reference material for atline injections) was injected onto a HIC column (example: Sepax Technologies, Inc.'s column Proteomix® HIC-Butyl 4.6×100 mm or Thermo Fisher Scientific's column MAbPAC™ HIC20 4.6×100 mm with a loading of 20 μg-100 μg.

Mobile phase A was 5 mM sodium phosphate, 1.5M ammonium sulfate, pH 6.9 with 2% ACN and mobile phase B was 5 mM sodium phosphate, pH 7.0 with 2% ACN with a column temperature of 30° C. For the Proteomic HIC Butyl column, the flowrate was 0.3 mL/min, the gradient used was: 50% B to 100% B from 0.0-20.0 min; 100% B from 20.0-21.0 min; and 100% B for 21.1-25.0 min. For the MABPAC HIC20 column, the flowrate was 0.5 mL/min, and the gradient used was: 20% B for 0.0-2.0 min; 100% B for 2.0-25.0 min; 100% B for 25.0-30.0 min; and 20% B from 30.1-35.0 min. The wavelength used for the PDA detector was 280 nm. The on-line PAP samples were injected approximately every 24 hours (a blank on-line injection and a blank at-line were repeated consecutively in between each on-line PAP injection similar to a negative control, in addition to help cleaning of the column).

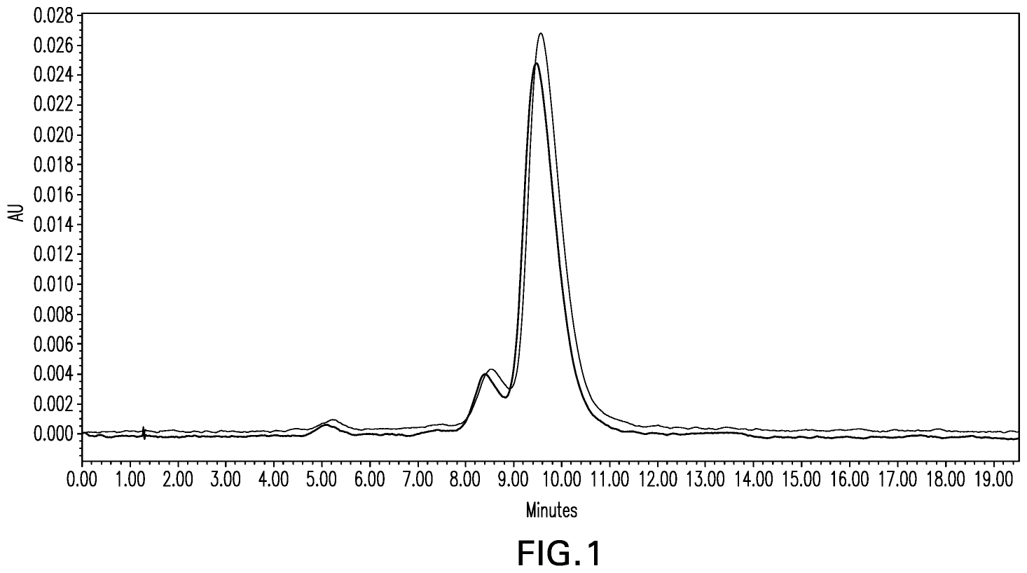

A HIC profile of an IgG4 mAb using the separating conditions as described above (without induced oxidation) is shown in FIG. 1. FIG. 1 displays a HIC profile of mAb 1 reference standard using the separating conditions as described in Example 1 via an atline or online injection.

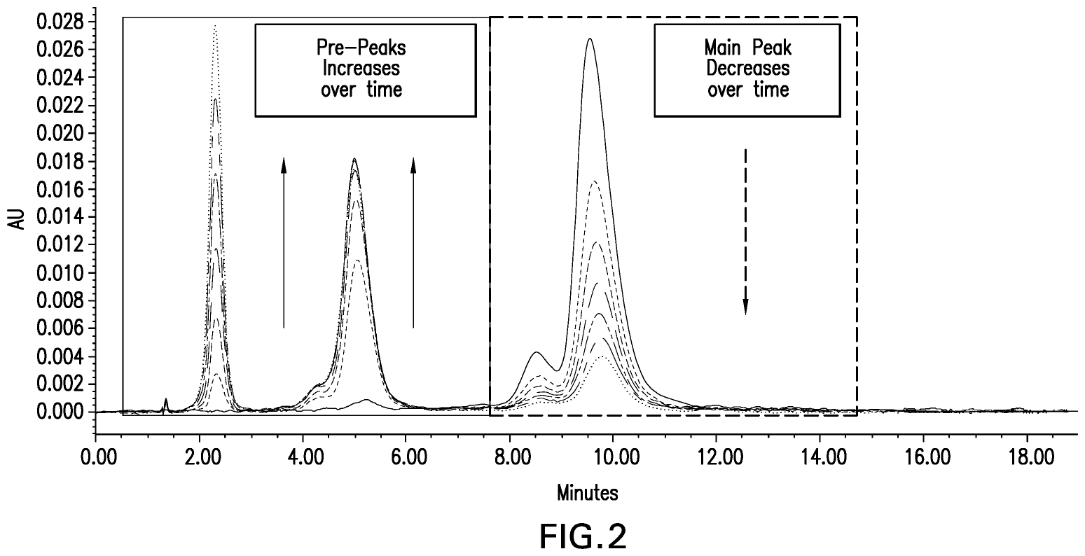

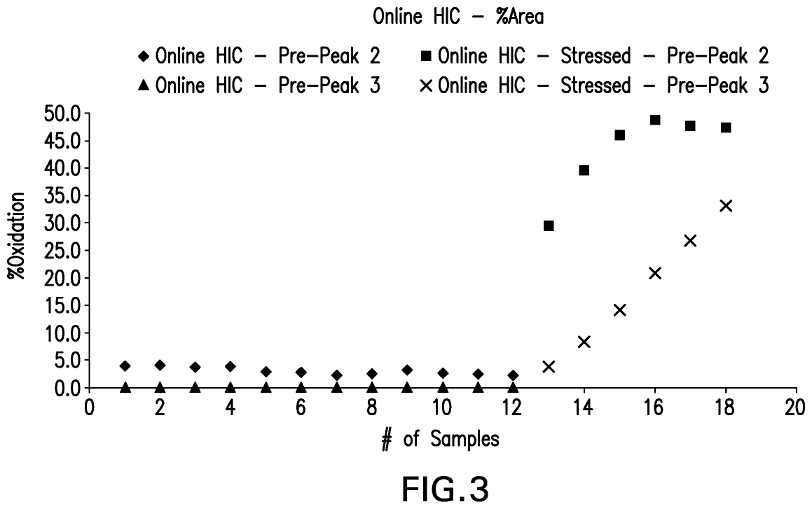

The product was intentionally oxidized by t-BHP, where the level of oxidization increases over time. FIG. 2 displays a HIC profile of t-BHP stressed mAb 1 using the separating conditions as described in Example 1. As shown in the graph, the t-BHP induced oxidation species were separated as pre-peaks. Quantitation of area percentage of pre-peaks correlated well with the orthoganol analysis via reduced peptide mapping. This correlation demonstrated that pre-peaks are oxidized species of mAb 1. The HIC profile dictates an IgG4 monoclonal antibody tested at various time points showing a change in oxidation levels with an increase in both pre-peaks, and a decrease in the main peak. As shown in FIG. 2, oxidation was induced in this example demonstrate method capability. The t-BHP oxidized population was separated as pre-peaks, which were further utilized for quantitation of oxidation (FIG. 3). FIG. 3 shows the samples tested for Example 2 for percent oxidation using an online HIC method with the control mAb 1 being t-BHP stressed according to Example 1. The results again show the increase of pre-peak 2 and 3 due to increasing levels of oxidation. In FIG. 3, the first two pre-peaks increase over time, while the main peak decreases over time. The t-BHP specifically oxidizes solvent exposed methionine residues, which correlates with an increase in only the pre-peaks indicating these pre-peaks have methionine species. The results from FIG. 2 and FIG. 3 demonstrate that once induced oxidation occurs, the percent oxidation rises in pre-peak 2 and 3 as obtained by online HIC.

### Example 2-15 Day Experiment

Example 2 was performed the same as Example 1 with the exception that the experiment was conducted over a 15 day period.

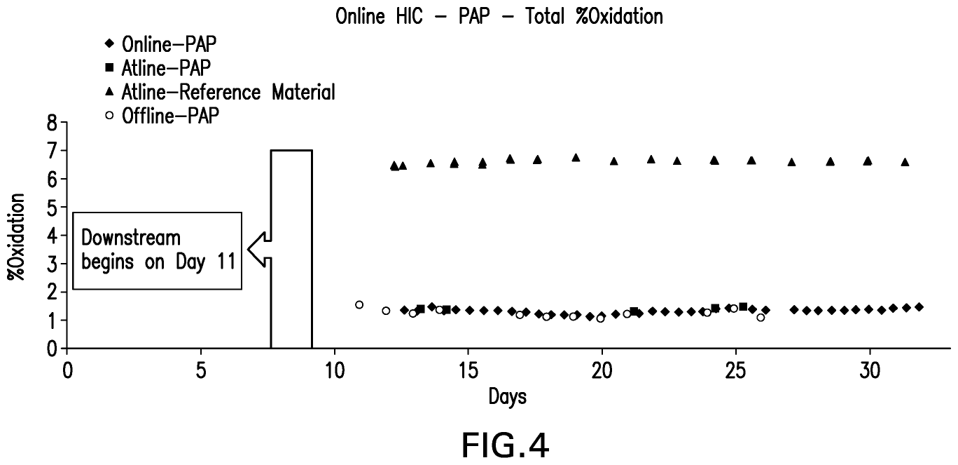

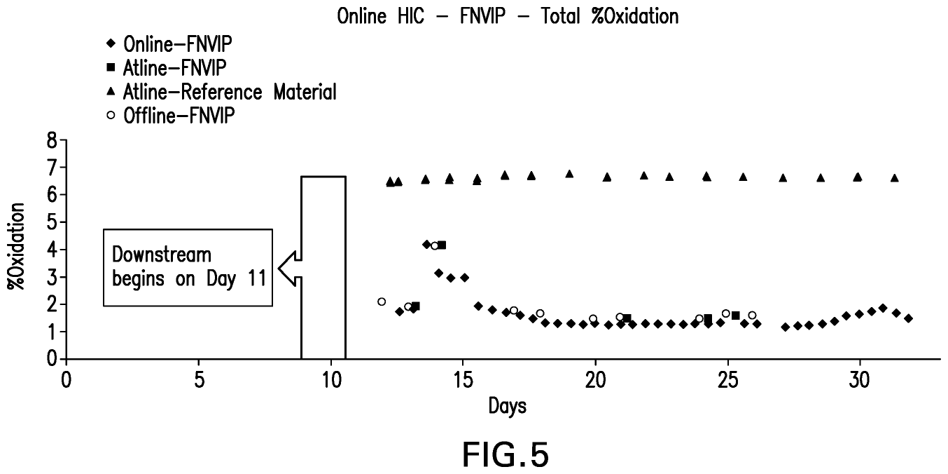

The Waters Patrol system was sampling both Protein A Product (PAP) and Filtered Neutralized Viral Inactivation Product (FNVIP) through its online capabilities to measure oxidation for over 15 days. During this process, the oxidation levels were steady and consistent for PAP (FIG. 4) (online, atline, and offline) while there was a change in oxidation between Day 10 and Day 15 for FNVIP (FIG. 5) and remained consistent afterwards. FIG. 4 shows total oxidation percent for Protein A product during this process, the oxidation levels were steady and consistent throughout the purification process (>20 days). The online sample measurements with the patrol are diamonds (♦), the atline sample measurement are squares (▪) while the atline standard measurements are triangles (▴). The offline measurement was performed on a separate benchtop UHPLC, which are circles (●). Note: the downstream purification process begins on day 11. FIG. 5 shows the oxidation level for filtered neutralized viral inactivation product. Note, the downstream process begins on Day 11. The online measurement with the Patrol is diamonds (♦), the atline measurement is squares (▪) while the atline measurement of the standard, which is the reference material, is triangles (▴). The offline measurement was performed on a separate benchtop UHPLC, which is circles (●). Note: the downstream process begins on day 11.

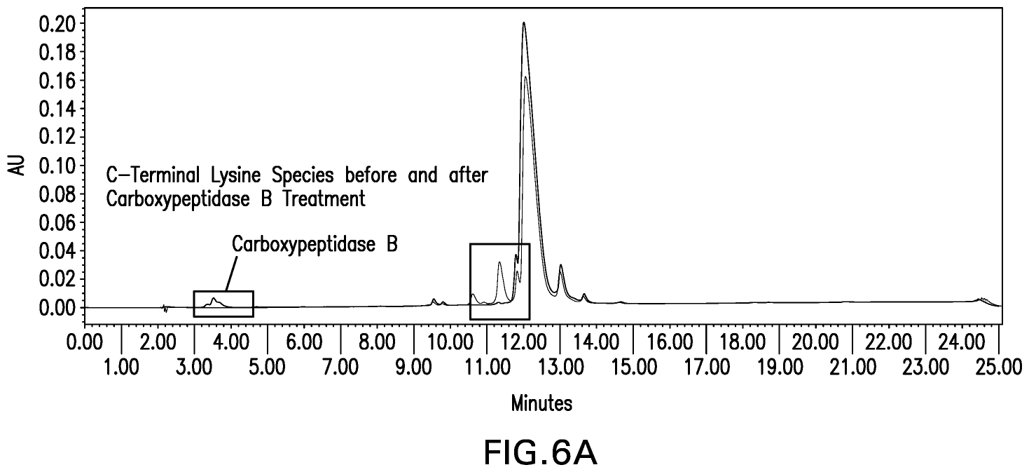

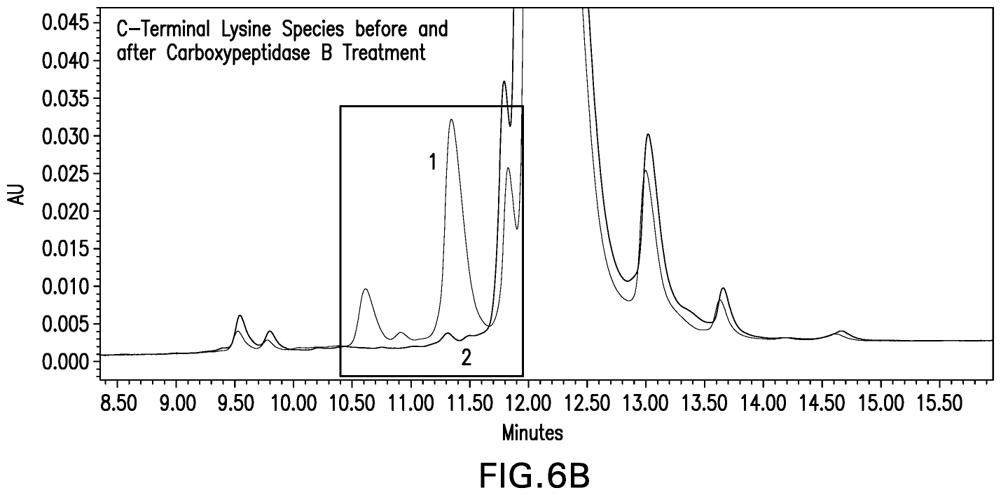

FIGS. 6A and 6B provide overlays of offline injections displaying the presence and absence of C-terminal lysine species. It highlights the HIC method capability in terms of separation and quantitation of species with C-terminal lysine, with no interference from quantitation of oxidation level. FIG. 6A displays an overlay of injections showing presence and absence of C-terminal lysine species before and after Carboxypeptidase B treatment (CpB) for an IgG4. Carboxypeptidase B is a proteolytic enzyme capable of rapidly hydrolyzing peptide bonds to release certain carboxyl-terminal basic amino acids from peptides and proteins. The first peak on the chromatogram is carboxypeptidase B added during sample preparation. FIG. 6B shows an overlay of injections displaying different levels of C-terminal lysine. FIG. 6B is a zoomed in image of FIG. 6A. The top line (‘1’) is representative of an injection showing the presence of C-terminal lysine species before Carboxypeptidase B treatment. The bottom line (‘2’) is representative of an injection showing the absence of C-terminal lysine species after carboxypeptidase B treatment.

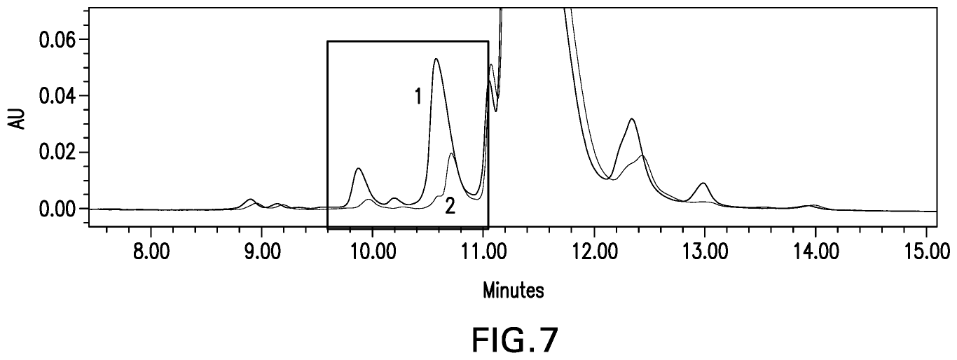

FIG. 7 shows an IgG4 generated under different upstream cell culture conditions displaying high levels of C-terminal lysine species (as represented by “1” in the figure) or lower levels of C-terminal lysine species (as represented by “2” in the figure) as detected by the HIC separation.

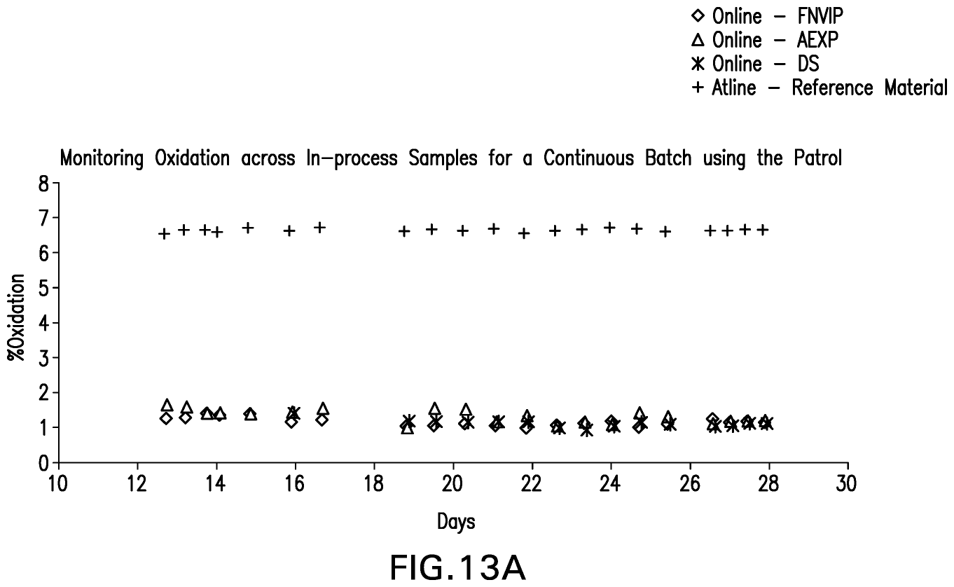

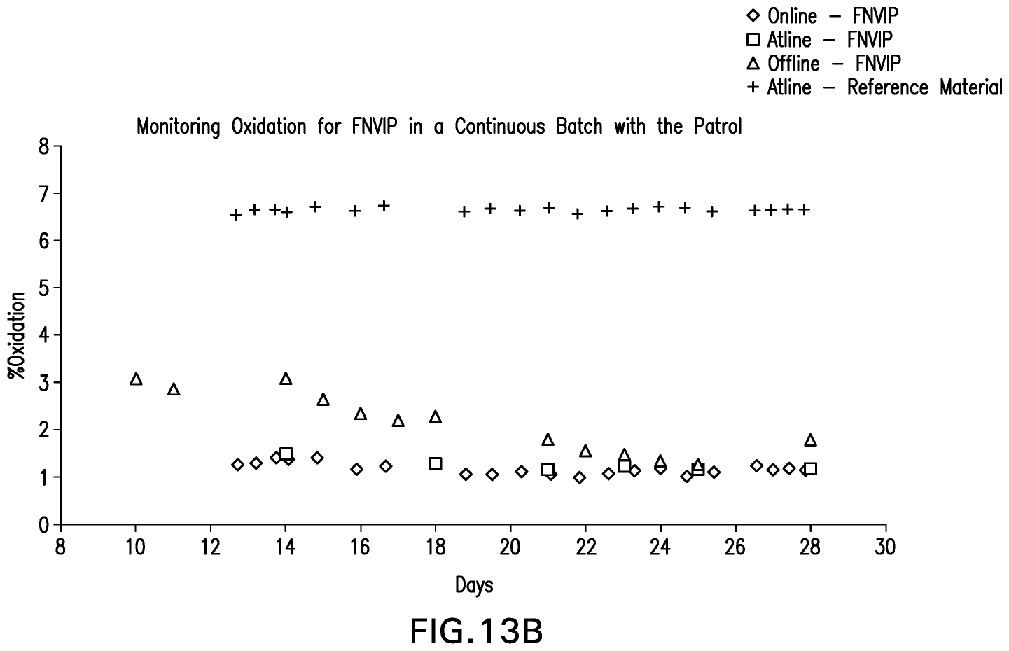

FIGS. 13A and 13B represent the on-line LC system measuring oxidation for multiple unit operations during a continuous batch in real-time. These results are compared to offline measurements, which show higher oxidation levels since they can potentially degrade over long periods of time. FIG. 13A shows the oxidation level for Filtered Neutralized Viral Inactivation Product (FNVIP), Anion-Exchange Product (AEXP), and Drug Substance (DS) along with the Reference Material using the HIC method with the Patrol during a continuous batch. The FNVIP, AEXP, and DS samples were analyzed as On-line injections, while the Reference Material was analyzed as an At-line injections. During this downstream process, there was no apparent change in the oxidation levels for each sample between day 12 and day 28. The online measurements for FNVIP with the patrol are yellow diamonds (♦), the online measurements for AEXP are purple triangles (▴), the online measurements for DS are red asterisks (*) while the at-line measurement of the reference material, are light purple crosses (+). FIG. 13B shows the oxidation level for Filtered Neutralized Viral Inactivation Product (FNVIP). During this downstream process, there was no apparent change in the oxidation levels for each sample between day 12 and day 28 for the on-line and at-line samples. The offline samples had a higher oxidation value due to the samples being tested at a later date, while the on-line samples were tested in real-time. The online measurements with the patrol are yellow diamonds (♦), the at-line measurement are blue squares (▪), while the at-line measurement of the reference material, are light purple crosses (+). The Offline measurement was performed on a separate benchtop UHPLC, which are teal triangles (▴).

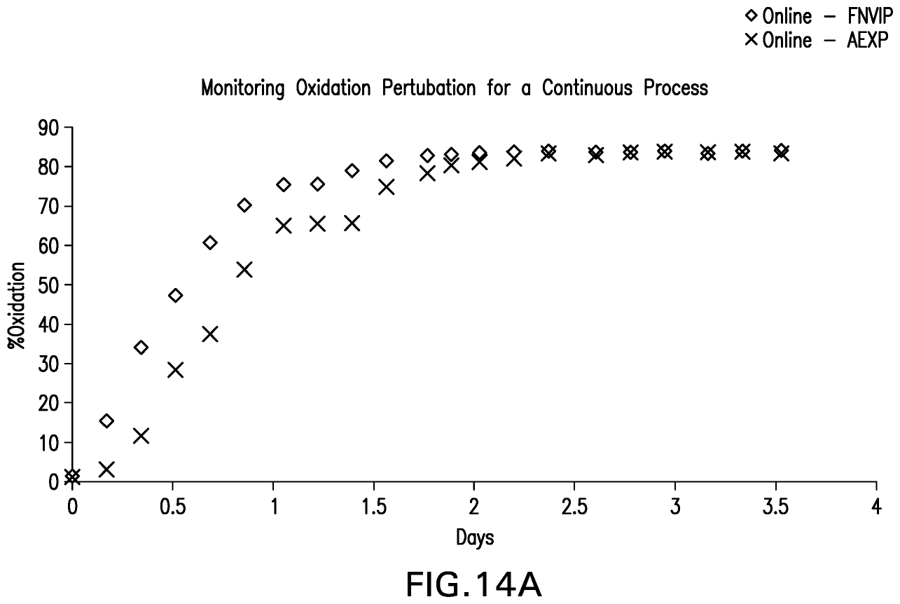

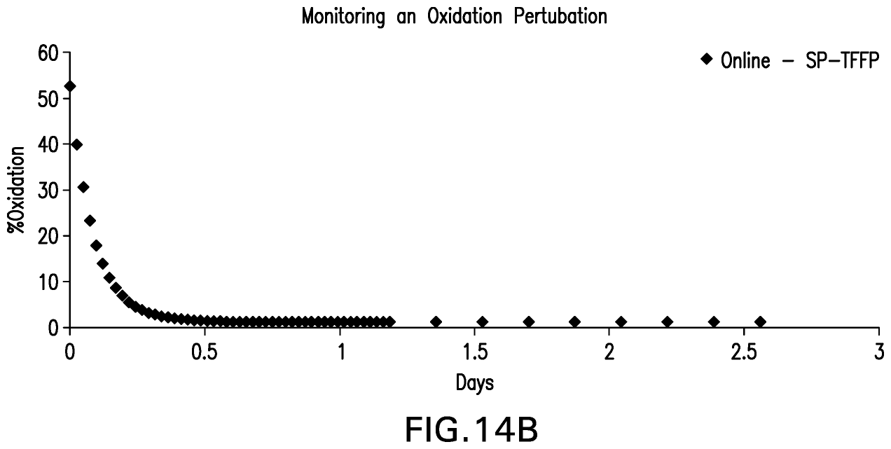

FIGS. 14A and 14B show the on-line LC system measuring a perturbation experiment where oxidized and non-oxidized material were being exchanged through various unit operations. FIG. 14A shows the oxidation level for Filtered Neutralized Viral Inactivation Product (FNVIP), and Anion-Exchange Product (AEXP). The FNVIP, and AEXP samples were analyzed as On-line injections. The Patrol was monitoring the changes in oxidation as non-oxidized material was being exchanged with oxidized material. This was being measured through the HIC method. The online measurements for FNVIP with the patrol are black diamonds (♦), the online measurements for AEXP are pink x's (x). FIG. 14B shows the oxidation level for Single Pass-Tangential Flow Filtration Product (SP-TFFP). The SP-TFFP samples were analyzed as On-line injections. The Patrol was monitoring the changes in oxidation as oxidized material was being exchanged with non-oxidized material. This was being measured through the HIC method. The online measurements for SP-TFFP with the patrol are red diamonds (♦).

### Example 3—On-Line 2D ProA-HIC to Measure Oxidation

To perform on-line two-dimensional (2D) Protein A-HIC (ProA-HIC) the Modular Automated Sampling Technology (MAST) system communicates to both the Gilson automated liquid Handler using Trilution software and the Agilent 1290 2D-LC using Chemstation software. The MAST platform can collect samples from one location and deliver that sample to one piece of equipment, such as Nova BioProfile FLEX, MAST Cell Removal System, or a Gilson Liquid Handler. The MAST system uses a sample pilot to aseptically sample from the connected continuous antibody production process and deliver the sample to the Gilson liquid handler. The sample pilot can collect and deliver the sample from the upstream or downstream cell culture process. The MAST system can sample from multiple locations including the bioreactor, which has the cells removed by the MAST Cell Removal System (CRS), or from a surge vessel after perfusion without the need for manual intervention. The Gilson liquid handler system collects the sample in a vial, and then redraws part of this sample from the vial to inject into a loop connected to a valve on the 2D-LC.

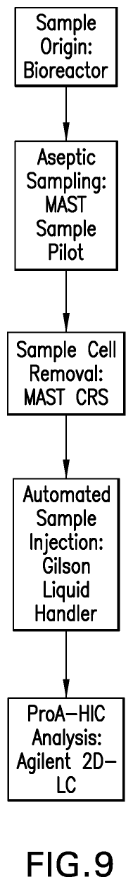

In this example, the MAST system automatically sends a signal to initiate a method created in Chemstation for the Agilent 2D-LC. Under control of Chemstation, the 2D-LC performs the ProA-HIC 2D separation using a fixed loop injection to measure the titer and oxidation of the antibody in the sample taken from the product stream. This sample undergoes the protein A chromatography step and then runs HIC on the sample. This is the first instance of automated on-line 2D-LC chromatography to measure oxidation of an antibody from bioreactor or harvested cell culture fluid. FIG. 9 is a flowchart representative automated workflow for on-line ProA-HIC analysis of bioreactor sample. FIG. 9 shows an aseptic sample is taken from the bioreactor using the MAST sample pilot, the cells are removed by the MAST cell removal system (CRS) before the sample is collected by the Gilson liquid handler, and that the sample is then automatically transferred to the Agilent 2D-LC for on-line ProA-HIC analysis.

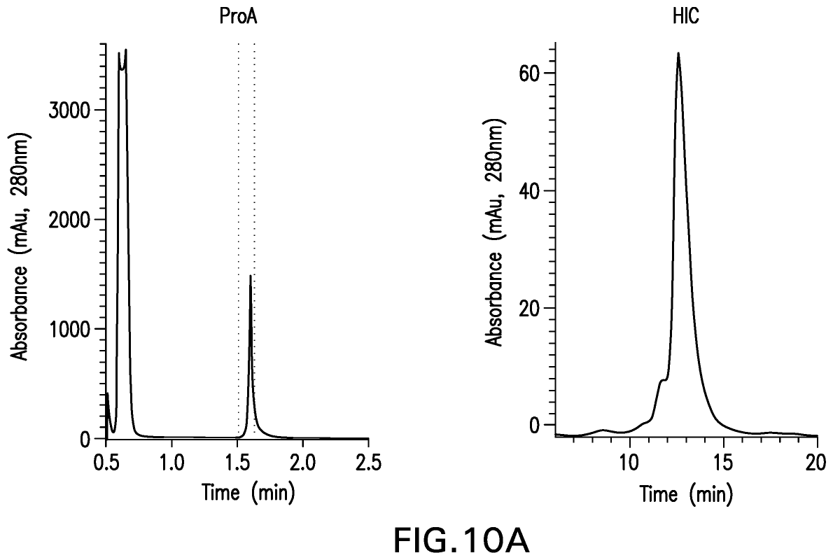

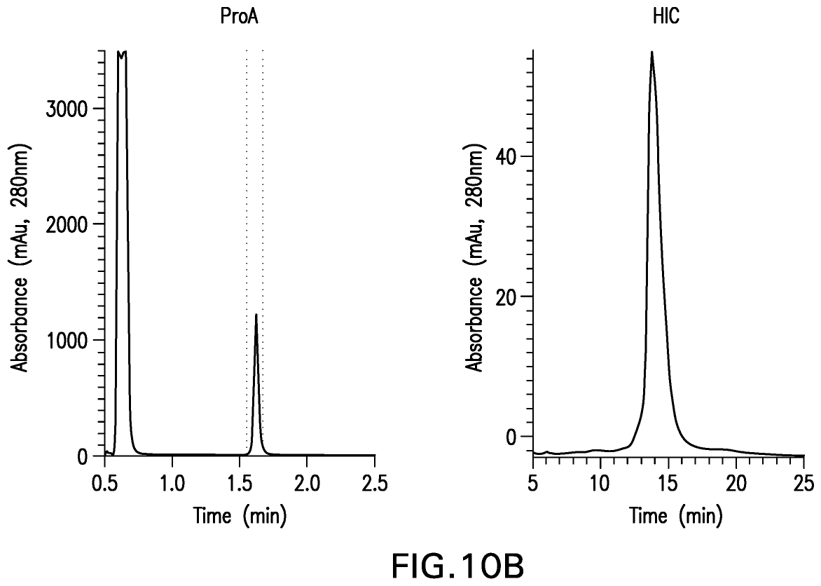

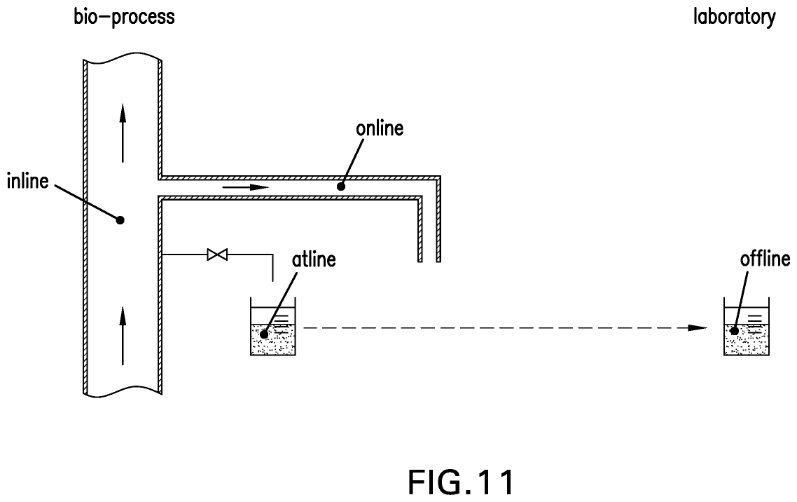

FIG. 10A provides two chromatography profiles showing an on-line analytical Protein A-HIC (ProA-HIC) chromatography 2D process to detect oxidation species from protein A feed. The dashed lines in the ProA separation show the fraction that was transferred to the second dimension (²D) for analysis by HIC. This data shows approximately 1% oxidation present in the Protein A feed. FIG. 10B shows a comparable set of data using a sample from the bioreactor after the cells were removed by the MAST CRS. FIG. 10B shows an on-line analytical ProA-HIC to detect oxidation species from bioreactor samples. The cells were removed from the sample before analysis by the MAST cell removal system (CRS). The dashed lines in the ProA separation show the fraction that is transferred to the second dimension (²D) for analysis by HIC. This data shows approximately 1.5% oxidation.

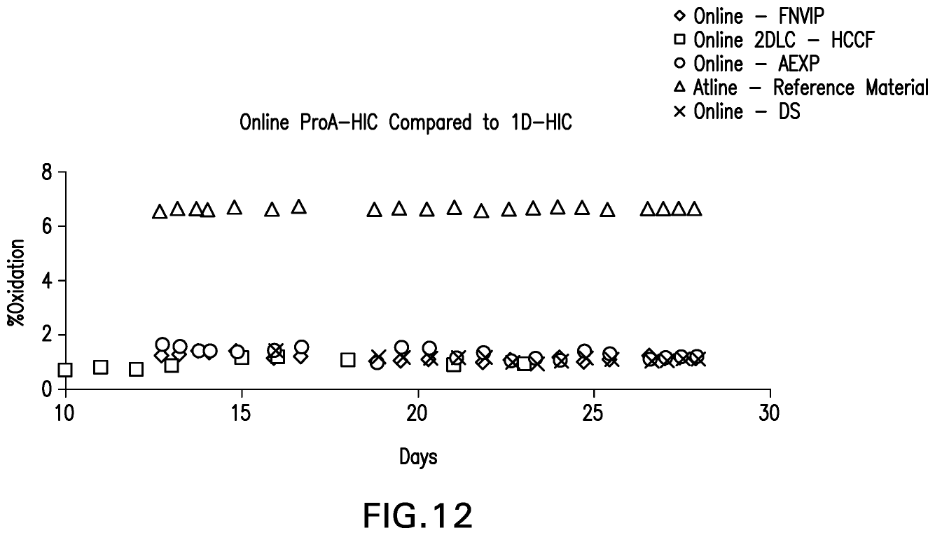

FIG. 12 shows the percent oxidation of samples taken during a continuous manufacturing process. The oxidation levels were steady and consistent between online 2D-LC analysis of the HCCF and 1D-LC analysis of later process intermediates. The online 2D-LC measurements are squares (▪). The online 1D-LC measurements are diamonds (♦), circles (●) and crosses (X) for FNVIP, AEXP, and DS, respectively. The atline reference material measurements are triangles (▴). FIG. 12 shows that the data from the online 2D-LC method is comparable to the online 1D-LC data collected from the same continuous manufacturing process.

### Example 4—Evaluation of Aggregation and N-Terminal Pyroglutamine of mAbs Via HIC Method

Materials:

Material/Reagent Name, Company Brand Name, Part Number
Sodium Phosphate Monobasic Monohydrate, Fisher BioReagents, BP330-1
Sodium Phosphate dibasic heptahydrate, Sigma Aldrich, S9390-1KG
Ammonium Sulfate, Sigma Aldrich, A2939-1KG
L-Arginine monohydrochloride, Sigma-Aldrich, 11039-500
MAbPAC HIC20 column, Thermo Fisher Scientific, 088554

Buffers:
Buffers: 5 mM sodium phosphate, pH 7.0
Buffers: 5 mM sodium phosphate, 1.0M ammonium sulfate, pH 6.9
Buffers: 50 mM Sodium Phosphate, 450 mM Arginine mono HCl, pH 7.0

Monoclonal Antibodies:

Antibody (mAb 2, IgG4) was produced in CHO cells and purified via a purification process including Protein A chromatography.

HIC Chromatography Method:

An injection volume of 5 μL DS was injected onto a HIC column (example: Thermo Fisher Scientific's column MAbPAC™ HIC20 4.6×250 mm) with a loading of 20 μg-100 μg.

Mobile phase A (5 mM sodium phosphate, 1.0M ammonium sulfate, pH 6.9) and mobile phase B (5 mM sodium phosphate, pH 7.0) were used at a flow rate of 1 mL/min and a column temperature of 30° C. The gradient used was: 0% B from 0.0-3.0 min; 0% B to 100% B from 3.0-40.0 min, 100% B for 40.0-50.0 min, 100% B to 0% B for 50.0-50.1 min; 0% B for 50.1-58.0 min. The wavelength used for the PDA detector was 280 nm.

HIC profiles of mAb 2 with different aggregates levels using the separating conditions as described above is shown in FIG. 14A via offline injection, including mAb 2 monomer (0.04% of aggregates, top panel), mAb 2 with heat stress at 40° C. for 3 months (1.86% aggregates, middle panel), and mAb 2 with enriched aggregates (91.11% aggregates, bottom panel). Note that the aggregates were measured through a size exclusion chromatography (SEC) method (Waters Acquity BEH200 SEC, P/N 186005226).

To understand the separation of aggregates under HIC, a zoom-in profile of each panel is provided in FIG. 14A. It can be demonstrated that monomer of mAb 2 elutes in the main peak regions of the HIC profile. The aggregates of mAb 2 elutes in post-peak region of the HIC profile.

To confirm the peak identities, an offline LC-MS was utilized for the analysis of monomer (FIG. 14A, top panel) and aggregates (FIG. 14A, bottom panel). From the deconvoluted mass spectrum shown in FIG. 14B, the molecular weight of monomer could be confirmed based on the sequence and major glycosylation of mAb2 (top panel, FIG. 14B, 150 kDa). Meanwhile, the identity of aggregates has also been confirmed to be mostly dimer (bottom panel, FIG. 14B, ˜300 kDa). Therefore, it could be concluded that HIC method can be used to separate and quantitate aggregates.

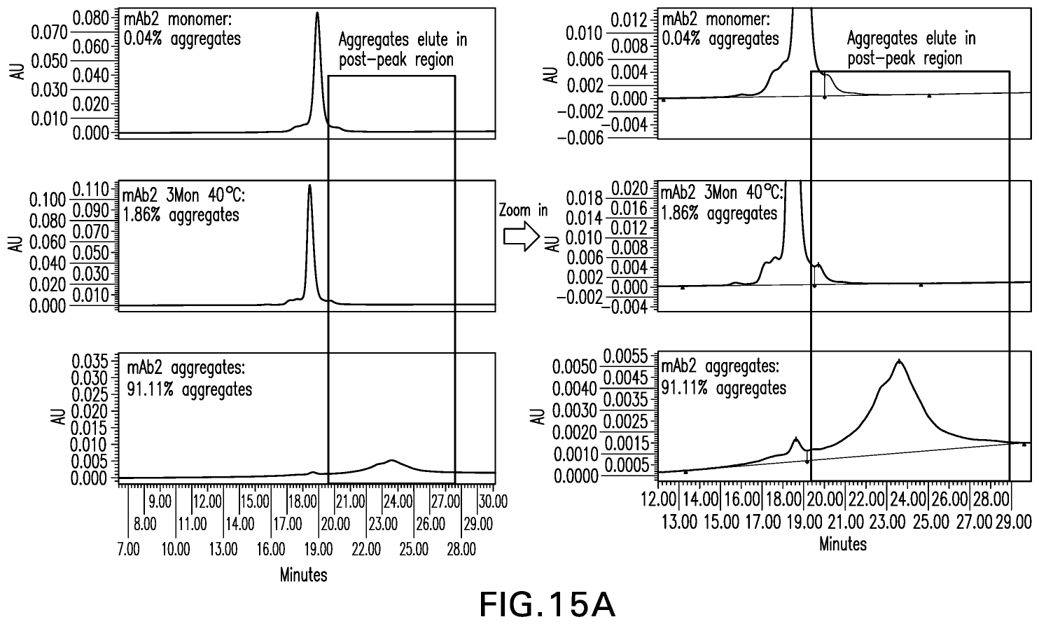

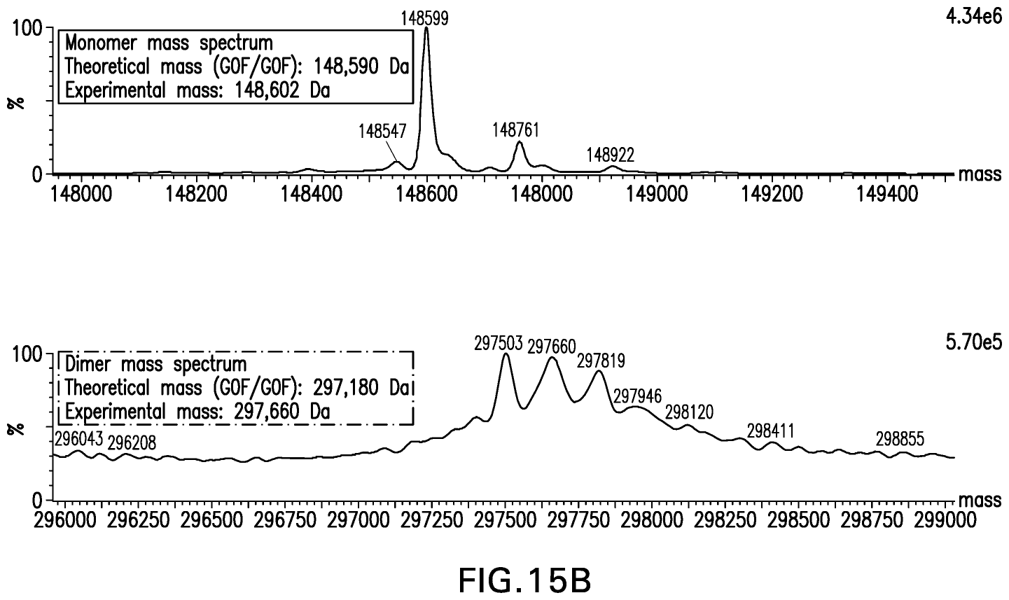

FIG. 15A shows the separation of aggregates (highlighted in the black box) via a HIC method. Top panel shows the HIC profile of mAb 2 monomer (only 0.04% aggregates), middle panel shows the HIC profile of stressed mAb 2 at 3 month 40° C., which contains 1.86% of aggregates. Bottom panel shows the HIC profile of enriched aggregates of mAb 2 collected from the stressed mAb 2 (HIC profile shown in the middle panel). The enriched aggregates contain 91.11% aggregates. Under HIC separation, the aggregates of mAb 2 elutes in the post-peak region. FIG. 15B shows the deconvoluted mass spectrum of mAb 2 monomer (top panel) and aggregates (bottom panel) to further demonstrate existence of aggregates (material profile shown in FIG. 15A bottom panel), and to confirm the identity of aggregates being dimer.

Isolation and Identification of N-Terminal Glutamine Cyclization

An injection volume of 5 μL DS was injected onto a HIC column (example: Thermo Fisher Scientific's column MAbPAC™ HIC20 4.6×100 mm) with a loading of 20 μg-100 μg.

Mobile phase A was 50 mM sodium phosphate, 1 M ammonium sulfate, pH 6.9 with 4% ACN and mobile phase B was 50 mM sodium phosphate, pH 7.0 with 4% ACN were used at a flow rate of 0.6 mL/min and a column temperature of 35° C. The gradient used was: 40% B from 0.0-3.0 min, 40% to 100% B from 3.0-18.0 min; 100% B from 18.0-20.0 min; and 100% to 40% B from 20.0-20.1 min, and 40% B from 20.1-25.0 min. The wavelength used for the PDA detector was 280 nm. Additionally, identical HIC method conditions were implemented to 2D LC-Ms (HIC-RP-MS) was utilized to support peak identity confirmation.

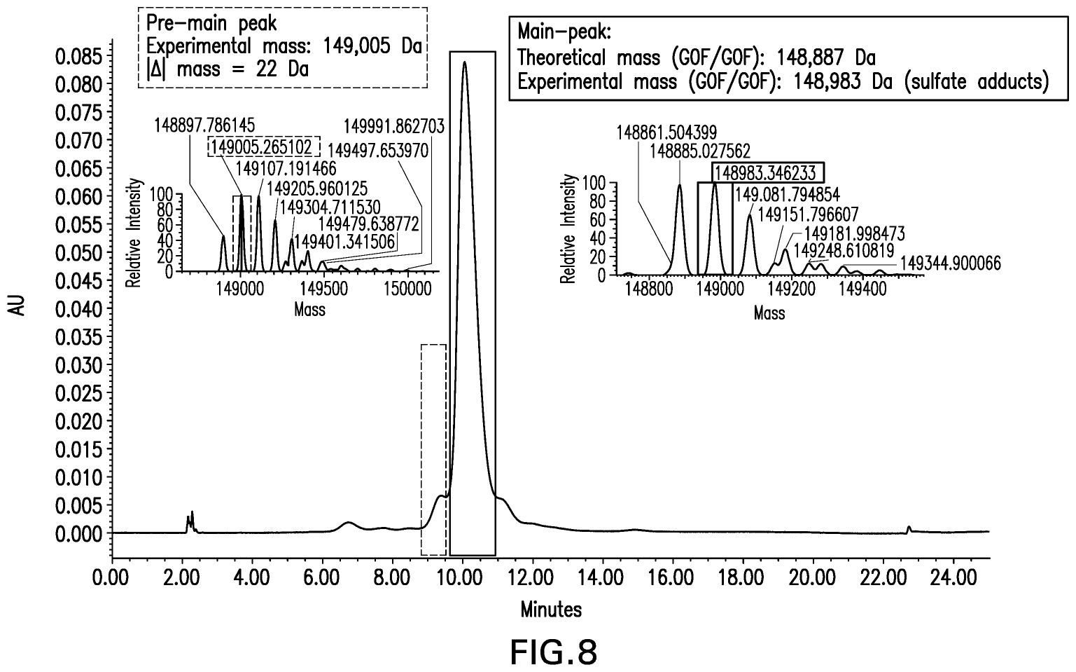

FIG. 8 provides identity of the pre-main peak of an offline injection via 2D-LC-MS (HIC-RP-MS) to be N-terminal pyroglutamine, while the main peak being N-terminal pyroglutamate, with experimental mass difference to be −22 Da, compared to theoretical mass difference of −17 Da.

1. A method of measuring at least one product attribute of a monoclonal antibody process intermediate by an online liquid chromatography system, wherein the liquid chromatography system is running a hydrophobic interaction chromatography (HIC) method, wherein the liquid chromatography system is connected to the purification process aseptically and operated in a continuous manner, and wherein the method comprises
(a) obtaining a sample from the purification process,
(b) automatically injecting said sample onto a hydrophobic interaction chromatography column,
(c) performing a real-time analytical method measuring at least one product quality attribute level,
(d) repeating steps a) and b) for the duration of the purification process, while analyzing and completing real-time process decisions to continue, stop, or segregate the downstream purification process.

2. The method of claim 1, wherein the product attribute is oxidation.

3. The method of claim 1, wherein the product attribute is aggregation.

4. The method of claim 1, wherein the product attribute is a post translation modification.

5. The method of claim 4, wherein the post translation modification is N-terminal glutamine cyclization and C-terminus lysine cleavage.

6. The method of claim 4, wherein the post translation modification is C-terminal lysine.

7. The method of claim 1, wherein the process intermediate is selected from the group consisting of: protein A feed, protein A product, viral inactivation feed, viral inactivation product, AEX load, AEX product, CEX load, CEX product, HIC load, HIC product, viral filtration feed, ultrafiltration feed, diafiltration feed, and bulk DS.

8. The method of claim 1, wherein the process intermediate is in the bioreactor.

9. The method of claim 1, wherein the process intermediate is protein A feed.

10. The method of claim 1, wherein the process intermediate is protein A product.

11. The method of claim 1, wherein the liquid chromatography system is connected to the purification process.

12. (canceled)

13. The method of claim 11, wherein the liquid chromatography system is connected to the purification process aseptically through a direct aseptic connection.

14. (canceled)

15. The method of claim 13, wherein the liquid chromatography system is operated in an integrated and continuous manner.

16. A method comprising an online hydrophobic interaction chromatography analytical method comprising the steps:
(a) obtaining a sample from the purification process,
(b) automatically injecting said sample onto a hydrophobic interaction chromatography column,
(c) performing a real-time analytical method measuring at least one product quality attribute level,
(d) repeating steps a) and b) for the duration of the purification process, while analyzing and completing real-time process decisions to continue, stop, or segregate the downstream purification process.

17. The method of claim 16, additionally comprising:
(a) optionally making process adjustments to the upstream bioreactor production process or downstream purification process based on the results obtained during real-time monitoring for the product quality attribute(s) subject to testing,
(b) wherein the process adjustments comprise continuing or stopping the downstream purification process, or segregating HCCF or the downstream product streams
(c) wherein segregating the HCCF or downstream product stream prevents contamination to HCCF or downstream product stream previously obtained prior to the process adjustment.

18. The method of claim 16, wherein the analytical method measuring at least one product quality attribute level comprises:
(a) obtaining a sample from the purification process,
(b) injecting a sample onto a hydrophobic interaction chromatography column;
(c) eluting based on salt concentration or pH;
(d) obtaining a value for a product quality attribute.

19. The method of claim 18, wherein the sample is a volume.

20. The method of claim 18, wherein the sample is a concentration.
